(19) United States
(12) Patent Application Publication
(10) Pub. No.: US 2026/0297557 A1
(43) Pub. Date: Oct. 1, 2026

(54) NUCLEIC ACID ISOLATION METHODS FOR USE IN MICROGRAVITY AND REMOTE ENVIRONMENTS

(71) Applicant: Christopher Mozsary, Brooklyn, NY (US)

(72) Inventor: Christopher Mozsary, Brooklyn, NY (US)

(21) Appl. No.: 19/475,622

(22) PCT Filed: Apr. 13, 2024
(86) PCT No.: PCT/US2024/024526

§ 371 Date: Oct. 15, 2025

Related U.S. Application Data
(60) Provisional application No. 63/459,638, filed on Apr. 15, 2023.

(51) Int. Cl. C12N 15/10

(52) U.S. Cl. CPC C12N 15/1013

(57) ABSTRACT

Method of extracting nucleic acids from a sample suitable for use in microgravity environments encountered during space travel are disclosed herein.

### REFERENCE TO RELATED APPLICATIONS

This application claims priority of U.S. Provisional Application No. 63/459,638 filed on Apr. 15, 2023, the disclosures of which are incorporated herein by reference in their entirety.

### BACKGROUND

### Technical Field

The disclosures herein relate to nucleic acid extraction techniques and related devices. Specifically, the disclosures relate to methods for extraction of deoxyribonucleic acid (DNA), ribonucleic acid (RNA), and other polynucleotides from biological material using minimal or no electrically powered equipment and without the use of toxic, volatile solvents. The devices and techniques are suitable for use in a low-gravity/zero-gravity environment.

### State of the Art

As human space exploration evolves and becomes increasingly common, the need for understanding how the extreme environment of space affects physiology has grown. The impact of space travel on the human body can be investigated by studying dynamic elements of the human genome, such as epigenetic modifications and telomeres. The best analyte for this application is DNA. To understand how DNA is affected by conditions unique to space travel, a method for in situ nucleic acid extraction must be developed. To accommodate constraints presented by the space capsule environment and challenges introduced by a lack of gravity, details of new methods for extracting DNA in space are presented herein.

Nucleic acid sequencing is essential to identifying and characterizing known life forms. Understanding biological activity at a molecular level, including human physiology, relies on the availability of nucleic acid sequencing. Recent technological advancements have made sequencing methods portable, inexpensive, and easy to perform, allowing nucleic acid sequencing under difficult environmental conditions found in remote locations on earth. Sequencing deep in a cave, a remote rainforest, on a glacier, or in other situations wherein adequate physical space, electrical power, and more favorable environmental conditions are lacking is advantageous for many reasons.

Extraction techniques for nucleic acid sequencing in space present additional challenges unique to the conditions highly specific to space travel. Power and physical space are severely limited, and both must be conserved. Payload weight restrictions are strict on any spaceflight mission The ideal extraction method, therefore, would not use any electricity, preserving battery life for other support systems, particularly crew life-support systems. Bulky specialty lab equipment such as centrifuges, heating blocks, refrigerators or freezers are problematic. The ideal extraction method would, accordingly, be performed manually and at room temperature.

No hazardous or flammable chemicals can be used. In the event reagents are introduced into the capsule environment, they must be non-toxic in case of exposure to crew. Fire in a closed, oxygen rich environment found in a space capsule would be catastrophic. Consequently, flammable reagents are not permitted in any space capsule environment. No speciality lab equipment, including heating or refrigeration apparatus, should be used save weight and physical space. Thus, the ideal method would utilize non-toxic and non-flammable reagents.

Microgravity environments make liquid handling particularly difficult, and a system that contains liquid throughout the workflow is necessary. There are also biosafety considerations. When working with blood or other biological material, it is essential to maintain sample isolation from the capsular environment to eliminate the possibility of exposing crew to pathogens, reinforcing the need for a closed system for the extraction reaction steps to occur.

Regardless of these challenges, nucleic acid sequencing in the challenging conditions associated with space travel is important, if not critical. The advent of commercial space exploration has provided new opportunities to study the effects of space flight on the human body. Analysis of blood samples taken from astronauts returning from the International Space Station has shown that living in an extraterrestrial environment for a prolonged period leads to genetic mutations and epigenetic changes in humans. See Garrett-Bakelman, et al., “A multidimensional analysis of a year-long human spaceflight,” Science. 2019 Apr. 12; 364 (6436), incorporated entirely herein by reference. Missions currently contemplated, such as staffing a permanent facility on the lunar surface or sending humans to Mars, could easily last years. Consequently, the ability to monitor genetic mutations and epigenetic changes in real time is needed to fully understand the effects of expanded space exploration and colonization on humans, and to develop mitigation strategies against potentially harmful changes to an astronaut's genome.

Portable nucleic acid sequencing devices are the ideal tools to facilitate this monitoring and are capable of conveniently generating genomic and epigenetic data in microgravity. A sequencing device, however, requires sufficient amounts of adequately concentrated target nucleic acid material to deliver accurate results.

Techniques of nucleic acid extraction utilizing currently available equipment are known and work well, both inside and outside of a conventional laboratory. The conditions present in a crewed space capsule, however, are very different than those in any Earth-based location. For example, physical space is restricted, available electrical power is limited, strict weight limits are required, there is a heightened fire risk in any oxygen-rich cabin, and any toxins present a high risk to human astronauts living in a completely closed environment. Additionally, fluids do not flow and are not constrained in a microgravity environment. These and other conditions pose substantial, unresolved challenges for extraction of purified nucleic acid from a raw biological sample during crewed space travel.

Restrictions of physical space within a crewed capsule or cabin are substantial. There is minimal or no room to set up a workspace. Electrical equipment requires a power source and means to dissipate heat. Crewed space capsules suffer strict limitations on available electrical power and ambient temperature within the capsule must be kept within a narrow range. Centrifuges and heaters used in many current methods of nucleic acid extraction take up substantial space and generate heat which is not easily dissipated from the crew capsule in extra-atmospheric conditions. Flight logistics, including payload weight restrictions and the lack of a temperature/humidity-controlled space to store bulky, temperature-sensitive precision analytical equipment for days or weeks pre-launch impose additional constraints.

Many current nucleic acid sample extraction methods rely on the use of organic solvents which may be toxic, highly flammable, or both. Microgravity in close quarters greatly increases the possibility of human or equipment exposure to liquids; i.e., through contact with liquid droplets floating in the space capsule environment. Consequently, no potentially hazardous substance can be used. The use of flammable solvents in particular, such as ethanol and other volatile organic solvents, is also forbidden.

Many methods for extracting and purifying nucleic acids from raw biological material for use in sequencing are currently available. Regardless of the method used, however, all are labor-intensive and require specialty precision laboratory equipment, employ the use of toxic or flammable solvents, reagents that require cold storage, a heat source to incubate chemical reactions, an electrical source to power multiple phases in the nucleic acid extraction and purification workflow, etc. It has not yet been possible to extract human nucleic acid for sequencing in an extraterrestrial environment due to these and other requirements of current extraction protocols.

For at least the foregoing reasons, improved methods of nucleic acid extraction suitable for extraterrestrial use are needed to overcome the deficiencies discussed herein above.

### BRIEF SUMMARY

Disclosed herein are embodiments of simplified method of nucleic acid extraction.

In some embodiments, the nucleic acid is DNA. In some embodiments, the nucleic acid is RNA. In some embodiments, the nucleic acid is combined DNA and RNA.

Disclosed is a simplified method of nucleic acid extraction from a biological sample comprising steps collecting a biological sample; preparing the sample; lysing the sample to create a lysate containing a nucleic acid in the absence of an organic solvent; fixing the nucleic acid; washing the lysate from the fixed nucleic acid; and eluting the nucleic acid, wherein the collecting step, the preparing step, and fixing step, the washing step, and the eluting step are performed without the use of electrically powered equipment and in the absence of an organic solvent.

Disclosed is a method of nucleic acid extraction from a biological sample comprising steps lysing a biological sample containing a nucleic acid to form a lysate; diluting the lysate; transferring the lysate binding the nucleic acid to a paramagnetic bead; removing a first supernatant; washing the bound nucleic acid; transferring the bound nucleic acid; pelleting the bound nucleic acid; removing a second supernatant; resuspending the bound nucleic acid; and eluting the nucleic acid from the paramagnetic bead; wherein the collecting step, the lysing step, the fixing step, the washing step, and the eluting step are performed without the use of electrically powered equipment and in the absence of an organic solvent.

The foregoing and other features and advantages of the invention will be apparent to those of ordinary skill in the art from the following more particular description of the invention and the accompanying drawings.

### BRIEF DESCRIPTION OF THE DRAWINGS

FIG. 1 is a diagrammatic representation of a simplified method of nucleic acid extraction;

FIG. 2 is a diagrammatic representation of an alternative embodiment of a simplified method of nucleic acid extraction;

FIG. 4A is an illustration of a diluting step;

FIG. 4B is an illustration of a first transferring step;

FIGS. 5A-B are illustrations of a binding step;

FIG. 6 is an illustration of a first removing step;

FIG. 7 is an illustration of a washing step;

FIG. 8 is an illustration of a second transferring step;

FIG. 9 is an illustration of a pelleting step;

FIG. 10 is an illustration of a second removing step;

FIG. 11 is an illustration of a resuspending step; and

FIG. 12 is an illustration of an eluting step.

### DETAILED DESCRIPTION

Methods for extracting DNA in space are presented here, including several illustrative embodiments of a chemistry workflow and accompanying apparatus.

As noted above, there are substantial limits to nucleic acid extraction techniques imposed by the space capsule environment. Electricity must be conserved—the ideal extraction method would not use any electricity to preserve battery/life support systems. The methods disclosed herein employ no hazardous or flammable chemicals. The disclosed reagents are non-toxic-should the disclosed reagents be introduced into the environment, human occupants would not be harmed. Further, the disclosed reagents and other materials are not flammable, given that combustible material is not permitted in the space capsule environment.

The nucleic acid extraction methods disclosed herein solely utilize non-toxic and non-flammable reagents. The disclosed methods require no specialty lab equipment, no need for cold storage of samples or reagents, and no techniques utilizing input of external heat. Because space capsules tend to be small on the inside and cannot accommodate bulky specialty lab equipment, such as centrifuges, heating blocks, refrigerators, or freezers, steps of the disclosed methods are executed manually and at room temperature. Moreover, a lack of gravity makes handling of liquids difficult. Thus, the disclosed methods fully contain liquids throughout the workflow. Consideration of biosafety is also necessary. When working with a biological sample, such as blood, it is essential to keep the sample isolated from the cabin environment to eliminate the possibility of exposing crew to pathogens. The methods disclosed herein employ a closed system wherein the extraction steps occur.

Various example embodiments of a simplified nucleic acid extraction method and kit are described in detail below. The method avoids the use of toxic and/or flammable organic solvents, centrifugation, an external source of controlled heating, other flammable materials, and specialty reagents that require frozen storage.

### Definitions

As used herein, “nucleic acid” means a substantially purified material comprising any of deoxyribonucleic acid (“DNA”), ribonucleic acid (“RNA”), polynucleotides incorporating deoxyribose or ribose, oligonucleotides incorporating deoxyribose or ribose, and related nucleic acid polymeric molecules of any length substantially free of histones and other protein material, cell membranes and lipids, and other contaminants which would interfere with nucleic acid sequencing reactions. The source of the nucleic acid may be nuclear DNA or RNA, cytosolic DNA, cytosolic RNA, free extracellular DNA or RNA, ex-vivo environmental sources.

In the several example embodiments disclosed herein, the extracted nucleic acid is DNA. This is not, however, meant to be limiting. The extraction of nucleic acids other than DNA are considered to be included in the disclosures provided herein.

As used herein, “biological sample,” “microsample,” or “sample” means material containing a nucleic acid target for extraction and purification using the embodiments of the methods disclosed herein. Non-limiting examples of samples include blood, including capillary, venous, arterial, and extravascular sources such as a surface; any bodily fluid that can be absorbed by an absorbent material, such as saliva, sputum, sweat, urine, semen; soft tissue samples; biopsy specimens; stool; and environmental sources wherein a surface, a liquid, a small portion of a solid substance, or the like may be swabbed, “taped,” or otherwise treated to collect the a sample for extraction and purification.

As used herein, “nucleic acid extraction” means removing nucleic acid, i.e., DNA, from cells in a substantially pure state suitable for a workflow to prepare DNA for sequencing. Nucleic acid extraction additionally means separating the nucleic acid from fragments of cell structures such as cell membranes, mitochondria, nucleosomes, and the like, including the lipid components thereof. Extraction also includes removal of histones, histone proteins, and other nucleic acid associated proteins and proteinaceous material.

As used herein, “sample lysate” or “lysate” means a liquid suspension of material following disruption of cells, organelles, and associated organized cell-derived components of the sample before performing steps to separate the nucleic acid from other lipid and proteinaceous material and additional steps to purify the nucleic acid placing it in a condition suitable for sequencing in an automated sequencing device.

As used herein, “centrifugation” means a technique of separating substances that involves the application of centrifugal force to separate a denser compound of a mixture from a less-dense component of the mixture. Centrifugation is a commonly used laboratory technique known to those of skill in the art of nucleic acid sequencing.

As used herein, “organic solvent” means a volatile organic (carbon-based) compound that vaporizes at room temperature.

### DESCRIPTION OF CERTAIN EXAMPLE EMBODIMENTS

Embodiments of the disclosed methods are based on a simplified paramagnetic bead extraction technique but with significant modifications. These modifications confer many advantages over the prior art which are necessary for successful nucleic acid extraction in extraterrestrial and other challenging environments and remote locations. No bulky, sensitive specialty equipment requiring electrical power is needed. Similarly, no external heat source is needed. Because paramagnetic beads are used for separation of nucleic acids from the sample lysate, no powered centrifuge is needed. Basic, stable reagents are used, and no refrigeration is needed to keep any reagents cold or frozen. Samples do not need to be frozen after collection because they can be extracted immediately. All steps are manual without the use of any electrical devices.

No heat or cold chain is needed. An enzymatic reaction requiring a controlled source of external heat is not required for lysis. The entire method is carried out at room temperature and is safe for the strict temperature regulation of the space capsule/cabin environment. Enzymes are not used to create a cell lysate from the biological sample, avoiding the requirement for frozen storage at −20° C. All reagents and materials are stable and can easily be kept in long-term storage without the need for temperature regulation.

All reagents are non-flammable, and organic solvents are not required. Precipitation of DNA from the processed lysate is performed with a buffer composed of polyethylene glycol and salt and does not require ethanol or isopropanol. No toxic materials are used. The sample lysate is created using mechanical cellular disruption only and is performed using nontoxic materials. All reagents throughout the simplified extraction method are considered to be nontoxic to humans.

Because Triton-X is a detergent that is environmentally hazardous and an irritant in high concentrations, the concentrations used in this protocol are very low. Triton-X concentration used in this method are no greater than about 1% by volume and in very small total volumes no greater than about 200 microliters (μL). Experimentation with other detergents, including conventional dishwashing liquid soap, extracted nucleic acids at a purity and concentration similar to Triton X however the charged (ionic) surfactants found in common dish soaps were found to interfere with downstream applications by altering the electrochemical properties of nucleic acids in the sample. A biodegradable nonionic surfactant, such as Ecosurf®, can be used as an alternative to Triton X-100 for use in regions that have banned Triton X-100 due to concerns about ecotoxicity.

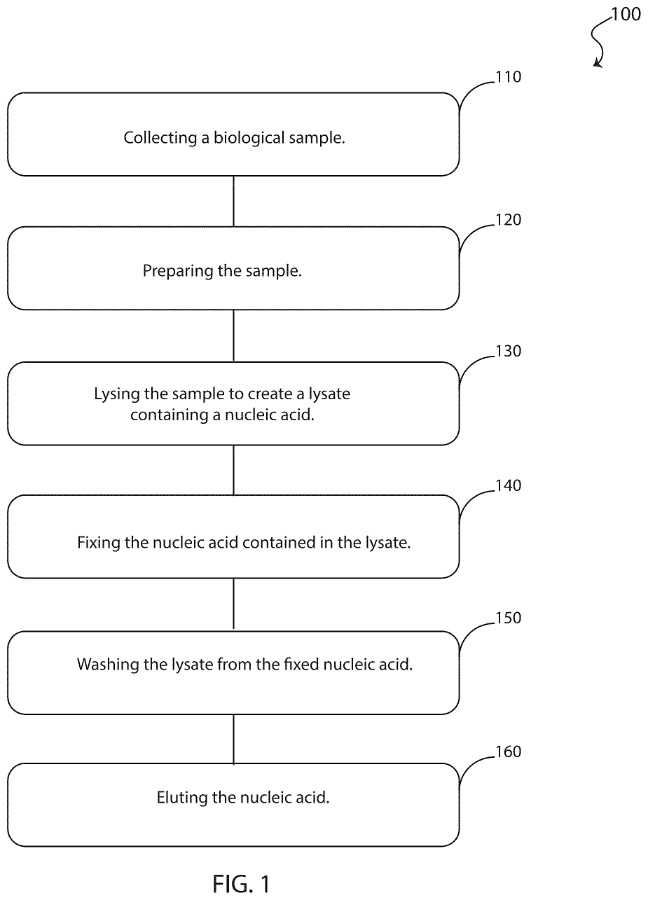

FIG. 1 is a diagram outlining the steps of a simplified method for nucleic acid extraction. FIG. 1 shows a method 100 comprising a collecting step 110, a preparing step 120, a lysing step 130; a fixing step 140; a washing step 150, and an eluting step 160. Method 100 is optimized for use in both closed-cabin microgravity environments and remote terrestrial environments where bulky equipment, electricity, temperature-sensitive reagents or reactions, and volatile or toxic materials are either not available or unsafe. Method 100 does not use enzymes or other reagents requiring storage under refrigeration, does not require organic solvents, does not require any devices or equipment that are powered by electricity, and does not require centrifugation.

Collecting step 110 comprises collecting a biological sample. Sample collection can be performed by any number of generally traditional means used for collecting a biological sample for nucleic acid extraction. In some embodiments, collecting step 110 includes collecting a capillary blood sample by pricking the skin, such as by using a lancet, a needle, or similar instrument, for example. A venous blood sample may be obtained by conventional venipuncture with blood collection into a standard Vacutainer® tube. However, a larger volume, i.e., equal to or greater than 1 mL. is not necessary to obtain a volume of DNA for sequencing using method 100. Collecting blood from a fingertip using a lancet typically yields a volume of blood of between about thirty (30) μL to about fifty (50) μL. Use of a blood sample in this volume range typically yields approximately 100 nanograms (ng.) of DNA+/−10% upon completion of method 100 and is considered optimal.

In some embodiments, collecting step 110 comprises using a lancet to pierce the skin and allowing blood to pool for about thirty (30) seconds to form a drop measuring about 1.0 centimeter in diameter on the fingertip. Massaging or milking the finger can be performed, if necessary, to facilitate pooling of an adequate amount of blood. Forceps or tweezers are used to contact the pooled blood with a piece of absorbent hydrophilic polymer foam measuring about 8 mm by 5 mm.

Other body fluids and samples may be obtained during collecting step 110, in some embodiments, including saliva, semen, and others. When saliva is used, DNA yields are typically lower than when extracting a blood sample of similar volume. In some embodiments, collecting the biological sample includes contacting a mucosa to obtain fresh cellular material for nucleic acid extraction, such as gently scraping the buccal mucosa with a wooden sample-collecting stick or rubbing with a cotton swab, for example.

In situations wherein method 100 is used in a microgravity environment, particular care must be exercised during collecting step 110 to prevent aerosolization, microdroplet, or droplet spread of blood into the cabin or capsule of the space vehicle. In some embodiments, this is facilitated by the use of a hydrophilic material as a carrier to transfer the blood droplet from the skin to an extraction vessel. Non-limiting examples of hydrophilic carriers suitable for use in step 110 include a polymeric foam such as the open-cell pyrolized carbon material incorporated by the Neoteryx® microsample collection device (Trajan Scientific Australia Pty. Ltd.), or a small piece of cellulose-based filter paper such as Whatman® filter paper. Using a small piece of filter paper or other hydrophilic carrier in collecting step 110 is advantageous because: (i) the carrier wicks away the blood sample while the blood droplet remains small enough to adhere to the skin by surface tension, thereby decreasing the chance that blood microdroplets will disperse into the cabin of an extraterrestrial vehicle under microgravity conditions; and (ii) cellulose fibers forming the filter paper aid in the mechanical disruption of intact cells, as described below. Experimentation comparing the use of Whatman® filter paper and a hydrophilic polymer foam demonstrates that use of the hydrophilic polymer foam as a means of collecting blood from a lanced fingertip and transferring it to the reaction vessel in step 110 results in higher yields of extracted DNA then when Whatman® filter paper is used.

Preparing step 120 comprises preparing the sample, in some embodiments. Preparing the biological sample includes placing the piece of filter paper, hydrophilic polymer, or other suitable hydrophilic carrier bearing the blood sample, sample-bearing material, or pure biological sample into a reaction vessel containing a lysing bead or a plurality of lysing beads and a quantity of lysis buffer. In some embodiments, a reaction vessel containing a plurality of lysing beads is used. In some embodiments, the plurality of lysing beads includes two, three, four, or greater than four lysing beads. Ideally, the reaction vessel is a residue-free small sterile container having a hydrophobic inner surface. In some embodiments, the reaction vessel is a 2 mL. microcentrifuge tube. In some embodiments, the reaction vessel is a syringe. One of skill in the art will appreciate that other similar devices may serve as the reaction vessel in preparing step 110 of method 100.

In some embodiments, a volume of blood collected by fingerstick or venipuncture as described herein above is drawn off the fingertip into a syringe via a needle or a pipet tip. In some embodiments, the hydrophilic material containing the sample is placed through the top of a syringe from which the plunger has been removed directly into the syringe barrel.

Lysing step 130 comprises lysing the sample to create a lysate containing a nucleic acid. Lysing step 130, in some embodiments, acts to lyse intact cells within the biological sample, denaturing histones and other nucleic acid-associated proteins, dissolving cell membranes and other lipid structures, separating the proteins and lipid structures from the nucleic acids, and aggregating the nucleic acid present in the sample. Lysing step 130 comprises chemical and mechanical disruption of cells and cellular structures to facilitate separation of DNA or other nucleic acids targeted for extraction from the sample. Chemical cellular disruption is facilitated by a lysing buffer. Mechanical disruption is facilitated by shaking or agitating the sample contained in the reaction vessel with one or more lysing beads.

Two steps to breaking down cellular or other biological matter for separation from nucleic acids are implemented in lysing step 130 of method 100 presented here. The first is a form of mild chemical lysis comprising treating the sample with a composition containing an ionic salt and a detergent. This partially disrupts cellular material and precipitates proteins, releasing nucleic acids into the aqueous lysis buffer. The second is mechanical disruption, which fragments biological matter for homogenization. The chemical mechanical treatments work synergistically to release nucleic acids from a biological specimen.

A common traditional method of chemical cell lysis utilizes a mix of phenol, chloroform and isoamyl alcohol to separate lipids and debris soluble in an organic phase from the water-soluble nucleic acids in an aqueous phase. Use of these compounds is unsafe for use in any small, confined space due to the increased potential for human exposure to the hazardous components of this reaction in zero gravity. Chemical cell lysis techniques using these components additionally requires centrifugation and refrigeration. In addition to being volatile and toxic, the reagents are also flammable. Consequently, use of these and other traditional reagents in lysing step 130 is contraindicated in lysing step 130 of method 100 which is practiced in a closed capsule/crew cabin environment present during space travel and extraterrestrial habitation. An alternative to this method is generally found in typical silica column extractions, which involves treating a biological sample with proteinase-K at a high temperature to break down proteins and free nucleic acids, then further treating with buffer formulations. As this method also relies on a hazardous protease, heating with specialty lab equipment and further processing the sample with a centrifuge, it is also not appropriate for a limited resource environment with the possibility of human contact. The ideal lysis method is one that uses no hazardous reagents or special instruments.

Note that the lysing bead and the lysis buffer are placed in the reaction vessel before adding the sample (i.e., blood or other fluid) or hydrophilic carrier bearing the sample during preparing step 120. A single standard spherical stainless steel lysing bead measuring between about four (4) millimeters (mm) and about six (6) mm in diameter is used, in some embodiments. Other materials may be used which do not oxidize, degrade, or otherwise change to potentially alter the composition of a high-osmolar salt solution used in method 100. Examples of such materials include glass, ceramic, and stone. Lysing beads of other sizes and materials depending on sample characteristics and known to those of skill in the art may similarly be used.

Various compositions may be used to form the lysis buffer and some non-exclusive examples are discussed herein. In some embodiments, a non-ionic surfactant is used, such as Triton-X 100, for example. In some embodiments, the lysis buffer used in preparing step 120 and lysing step 130 comprises a solution of 1× phosphate-buffered saline (PBS), 500 millimolar (mM) NaCl and 0.5% Triton-X detergent in nuclease-free water. In some embodiments, the lysis buffer used in preparing step 120 and lysing step 130 consists of an aqueous solution of 4.5 molar (M) NaCl and 1% Triton-X detergent. A very small volume of lysis buffer is used, such as 200 μL., in some embodiments. Use of a small volume of high osmolality lysis buffer is also an important element of method 100. Placing cellular material in a high osmolar environment combined with a surfactant detergent, such as Triton-X, denatures nucleic acid-associated proteins while salt aggregates the nuclei acid, facilitating separation of the nucleic acid from proteins and other molecular species present in the lysate. Lower yields are obtained using method 100 with lower concentrations of Triton-X or other surfactant-containing detergents. Reduced lysis efficiency is observed in lower salt concentration. Additionally, the use of a very small volume (i.e., about 200 μL) allows room in the small reaction vessel for the subsequent addition of a larger volume of water or low-osmolality buffer to reduce the concentration of salt and detergent in the lysate.

In some embodiments of lysing step 130, sodium chloride is used to precipitate protein. NaCl can be diluted with PBS to reduce the NaCl “carryover” concentration downstream, but the efficiency of lysing step 130 is increased when no PBS is used and the NaCl solution is highly concentrated. Details are provided in the examples shown in Table 1 and Table 2 below. Table 1 summarizes an example PBS-based lysis buffer efficiency test. Absorption ratios of 260 nm/280 nm were measured to compare the resulting eluate from the PBS-based lysis buffer following combined chemical and mechanical lysis with two lysis beads in the reaction vessel gently agitated for approximately two (2) minutes. As used herein, “blood:LB ratio” means blood to lysis buffer ratio by volume. “BB duration” means the duration of mechanical agitation within the reaction vessel containing lysis beads.

TABLE 1
PBS-based lysis buffer efficiency test
PBS-based lysis buffer efficiency test
Testing A260/280 absorption ratios of eluate from PBS-based lysis buffer
2 min. bead beating (BB), 0.8x SPRI cleanup with 10 min.
incubations, PBS without wash resuspension

| | blood:LB | Blood | LB | | BB duration |
|---|---|---|---|---|---|
| Sample | ratio | V (uL) | V (uL) | Beads | (min) |
| A | 1:2 | 50 | 100 | Yes | 2 |

| Lysis buffer | Final | Vol | Wash buffer | Final | Vol |
|---|---|---|---|---|---|
| (LB) | C | (uL) | (WB) | C | (uL) |
| NaCl 5M | 0.5M, | 200 | NaCl 5M | 0.5M | 200 |
| Triton | 0.5% | 100 | PEG 3000 50% | 5% | 200 |
| X-100 10% | | | | | |
| PBS 1X | 85% | 1700 | H2O | | 1400 |

| Lysate | H2O | Diluted | SPRI | Wash | Elute |
|---|---|---|---|---|---|
| (uL) | (uL) | lysate (uL) | Beads (uL) | (uL) | (uL) |
| 100 | 900 | 800 | 640 | 1000 | 22 |

| | C | V | DNA | | |
|---|---|---|---|---|---|
| Sample | (ng/uL) | (uL) | (ng) | A260/280 | Appearance |
| A1 | 8.5 | 20 | 169.6 | 1.02 | Light pink |
| A2 | 9.3 | 20 | 186 | 0.98 | Light pink |
| A3 | 9.8 | 20 | 196 | 1.04 | Light pink |

Table 2 below summarizes nucleic acid yields to determine the efficiency of a high concentration NaCl-based lysis buffer that does not include PBS.

TABLE 2
NaCl-based lysis buffer efficiency test
NaCl-based lysis buffer efficiency test
Testing A260/280 absorption ratios of eluate from NaCl-based lysis buffer
2 min. bead beating (BB), 0.8x SPRI cleanup with 10 min.
incubations, resuspension in wash

| | blood:LB | Blood | LB | | BB duration |
|---|---|---|---|---|---|
| Sample | ratio | V (uL) | V (uL) | Beads | (min) |
| A | 1:2 | 50 | 100 | Yes | 2 |

| Lysis buffer | Final | Vol | Wash buffer | Final | Vol |
|---|---|---|---|---|---|
| (LB) | C | (uL) | (WB) | C | (uL) |
| NaCl 5M | 4.75M, | 1900 | NaCl 5M | 0.5M | 200 |
| Triton | 0.5% | 100 | PEG 3000 50% | 10% | 200 |
| X-100 10% | | | | | |
| | | | H2O | | 1400 |

| Lysate | H2O | Diluted | SPRI | Wash | Elute |
|---|---|---|---|---|---|
| (uL) | (uL) | lysate (uL) | Beads (uL) | (uL) | (uL) |
| 100 | 900 | 800 | 640 | 1000 | 22 |

| | C | V | DNA | | |
|---|---|---|---|---|---|
| Sample | (ng/uL) | (uL) | (ng) | A260/280 | Appearance |
| A1 | 10.8 | 20 | 216 | 1.52 | Clear |
| A2 | 11.2 | 20 | 224 | 1.49 | Clear |
| A3 | 11.6 | 20 | 232 | 1.54 | Clear |

Following placement of the sample in the reaction vessel, the reaction vessel containing the sample-carrier, lysis buffer, and lysing bead(s) is agitated to initiate mechanical disruption. In some embodiments, agitation comprises vigorously shaking the reaction vessel for about two minutes. Shaking the reaction vessel containing both the lysing bead and the hydrophilic carrier mechanically disrupts nucleic acid-containing cells within the sample.

Table 3 below shows the effect of using different volumes of lysis buffer in lysing step 130, in some embodiments.

TABLE 3
NaCl-based lysis buffer volume trials
Lysis buffer volume trials
Determine effect of lysis buffer (LB) volume on yield
2 min. bead beating (BB), 0.8x SPRI cleanup with
10 min. incubations, Resuspension during wash

| | Blood:LB | Blood V | LB V | | BB time |
|---|---|---|---|---|---|
| Sample | ratio | (uL) | (uL) | Beads | (min) |
| A | 1:2 | 50 | 100 | Yes | 2 |
| B | 1:4 | 50 | 200 | Yes | 2 |
| C | 1:1 | 100 | 100 | Yes | 2 |

| Lysis | | Vol | Wash buffer | | Vol |
|---|---|---|---|---|---|
| buffer (LB) | Final C | (uL) | (WB) | Final C | (uL) |
| NaCl 5M | 4.5M | 1800 | NaCl 5M | 0.5M | 200 |
| Triton X-100 | 1% | 200 | PEG 3000 | 10% | 400 |
| 10% | | | 50% | | |
| | | | H2O | | 1400 |

| Lysate | H2O | Diluted lysate | SPRI Beads | Wash | Elute |
|---|---|---|---|---|---|
| (uL) | (uL) | (uL) | (uL) | (uL) | (uL) |
| 100 | 900 | 800 | 640 | 1000 | 22 |

| | C | V | DNA | A260/ | |
|---|---|---|---|---|---|
| Sample | (ng/uL) | (uL) | (ng) | 280 | Appearance |
| A1 | 10.7 | 20 | 214 | 1.23 | Clear |
| A2 | 11.3 | 20 | 226 | 1.17 | Clear |
| A3 | 7.82 | 20 | 156.4 | 1.25 | Clear |
| B1 | 10.4 | 20 | 208 | 1.45 | Clear |
| B2 | 9.94 | 20 | 198.8 | 1.49 | Clear |
| B3 | 10.4 | 20 | 208.8 | 1.46 | Clear |
| C1 | 14.1 | 20 | 282 | 1.2 | Clear/hazy |
| C2 | 13.4 | 20 | 268 | 1.24 | Clear/hazy |
| C3 | 13.8 | 20 | 276 | 1.18 | Clear/hazy |

Fixing step 140 comprises fixing the nucleic acid contained in the lysate. In some embodiments, fixing step 140 comprises placing paramagnetic beads into the extraction vessel. Suitable paramagnetic beads include AMPure XP® paramagnetic beads made by Beckman Coulter Inc., Brea, California. Other examples of paramagnetic beads conventionally used for nucleic acid washing and extraction may also be employed in fixing step 140.

In some embodiments, paramagnetic beads specific to RNA extraction are used. In some embodiments, the reaction vessel is gently swirled for 3-10 seconds after addition of the paramagnetic beads and then allowed to stand undisturbed at ambient temperature for about ten (10) minutes. In some embodiments, the reaction vessel is rocked back and forth about 20 times. The reaction vessel is then placed in a magnetic field, such as by placing the microcentrifuge tube in a magnet stand (DynaMag-5, ThermoFisher Scientific Inc., Waltham, Massachusetts.) The reaction vessel remains in the magnetic field for about five (5) minutes, in some embodiments, to allow for pelleting of the paramagnetic beads.

Washing step 150 comprises washing the lysate from the fixed nucleic acid. In some embodiments, washing step 150 includes careful removal, such as by pipetting, of the supernatant from the pelleted paramagnetic beads followed by washing the pelleted beads with a solution of wash buffer. In some embodiments, the wash buffer comprises polyethylene glycol (PEG) and sodium chloride (NaCl). In some embodiments, the pelleted paramagnetic beads are washed twice with wash buffer. In some embodiments, the beads are washed by removing the magnetic field and resuspending the beads in the wash buffer, then returning the magnetic field to the solution to re-form a pellet, then removing the supernatant.

Eluting step 160 comprises eluting the nucleic acid. In some embodiments, eluting step 160 includes removing the reaction vessel from the magnetic field and then resuspending the paramagnetic beads in a solution of 1× Tris and EDTA buffer (TE buffer). The use of TE buffer instead of water preserves nucleic acids and mitigates nucleic acid degradation over time. The suspended beads are allowed to incubate at ambient temperature for about three (3) minutes, in some embodiments. The reaction vessel is then placed back in the magnetic field and the beads are allowed to re-pellet for about three (3) minutes, in some embodiments. With the reaction vessel in the magnetic field and without disturbing the pelleted beads, the supernatant is removed, such as by pipetting, and placed in a second clean, sterile tube or container containing the eluted, extracted nucleic acid.

In some embodiments, multiple second tubes of extracted nucleic acid, such as DNA, are combined into pooled aliquots. A combined aliquot can be sequenced or stored for later sequencing or analysis.

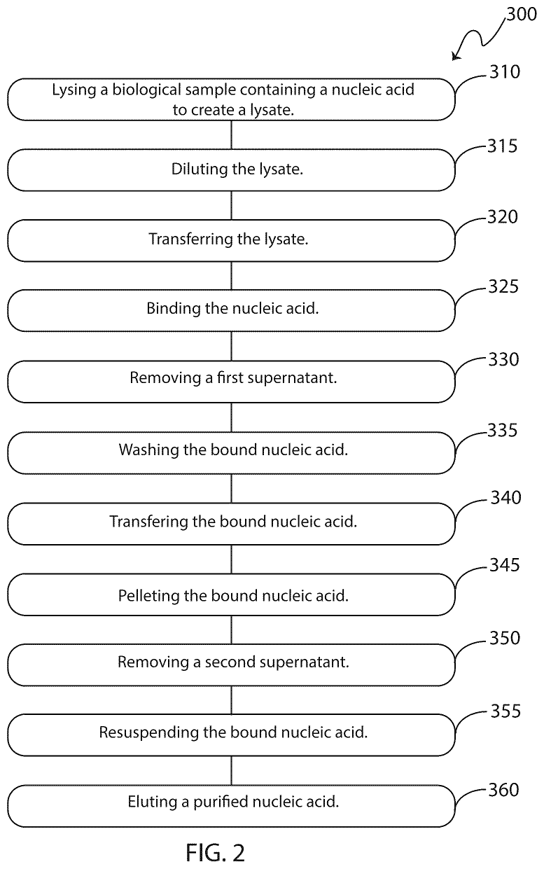

In situations wherein methods of nucleic acid extraction are used in a microgravity environment, particular care must be exercised during all steps to prevent aerosolization, microdroplet, or droplet spread of blood into the cabin or capsule of the space vehicle. Accordingly, FIGS. 2-11 show an example embodiment of a nucleic acid extraction method 300 and apparatus wherein the biological sample and all reactants are transferred between two coupled syringes in a quasi-closed system, whereunder lysing step 130, fixing step 140, washing step 150, and eluting step 160 of method 300 are all performed with the sample, lysis buffer, and sequential aliquots of washing buffer constrained between the first syringe and a second syringe used to add washing buffer and remove waste buffer. Method 300 does not use enzymes or other reagents requiring storage under refrigeration, does not require organic solvents, does not require any devices or equipment that are powered by electricity, and does not require centrifugation.

FIG. 2. is a diagrammatic representation of an alternative embodiment of a simplified method of nucleic acid extraction. As noted, handling of liquids in microgravity presents additional challenges. Liquids do not flow in response to gravity. Liquids will readily disperse through the volume of a container and float or drift out of a container open to the larger surrounding space. Consequently, in some embodiments, extraction reactions are performed in a largely closed system where liquid reactants are tightly constrained within the reaction vessel.

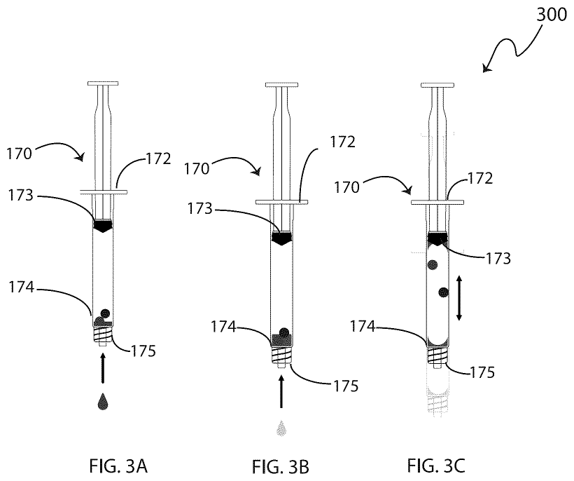

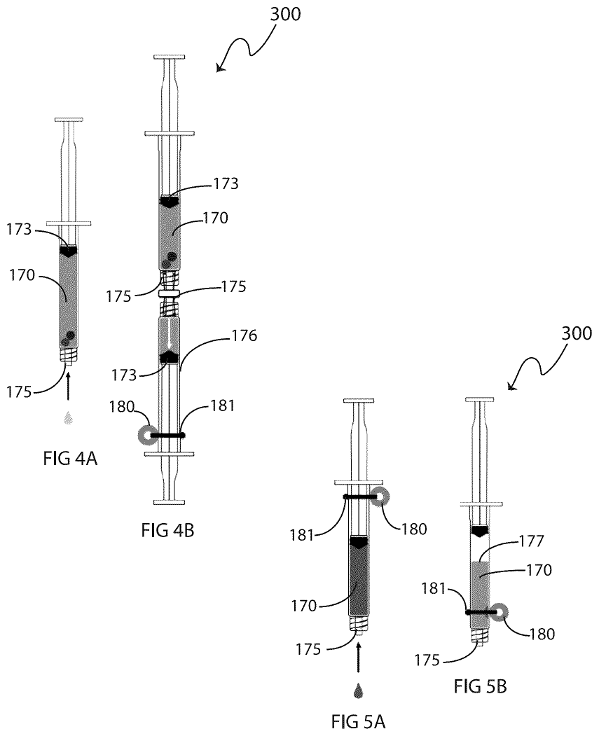

FIG. 2 shows steps of a method 300 for extracting nucleic acids from a biological sample. In some embodiments of method 300, a reaction vessel 170 is a syringe comprising a first end 172 closed by a plunger 173 and a second end 174 bearing a coupling fitting 175. A magnet 180 is slidably coupled to reaction vessel 170, such as to an outer wall of vessel 170 with a slider 181 as shown in FIGS. 4B-10. Embodiments of method 300 shown in FIG. 2 are depicted and the several method steps are discussed wherein reaction vessel 170 is a syringe.

As shown in FIG. 2, method 300 comprises a lysing step 310, a diluting step 315, a first transferring step 320, a binding step 325, a first removing step 330, a washing step 335, a second transferring step 340, a pelleting step 345, a second removing step 350, a resuspending step 355, and an eluting step 360.

Lysing step 310, in some embodiments, comprises lysing a biological sample containing a nucleic acid to create a lysate. FIGS. 3A-C show lysing step 310. A reaction vessel 170 is used. The biological sample is drawn into or placed within reaction vessel 170 containing a lysis bead or a plurality of lysis beads, as discussed previously herein. In some embodiments, for example, a volume of blood is drawn up into reaction vessel 170 via a needle, pipette tip, or added from a first end 172 (with a plunger 173 removed) in the form of soaked swabs or other carrier wherein the biological sample, such as blood from a fingerstick or venous blood draw, has been absorbed. A volume of lysis buffer is then drawn into vessel 170 and either a closure (not shown) or a transfer syringe 176 as shown in FIG. 4B the vessel 170 is shaken for about two (2) minutes to effect mechanical disruption of cellular components of the biological sample, as discussed previously herein.

Diluting step 315, in some embodiments, comprises diluting the lysate. FIG. 4A shows diluting step 315, wherein a volume of diluent, such as water, in some embodiments, is drawn through coupling fitting 175 into vessel 170. In some embodiments, the diluent is drawn directly from a transfer syringe (not shown) coupled to vessel 170 at a coupling fitting 175. FIG. 4A shows the lysate diluted with the diluent within vessel 170.

First transferring step 320, in some embodiments, comprises transferring the lysate. FIG. 4B shows first transferring step 320. A separation tube 176 also bearing coupling fitting 175 is coupled to vessel 170 at the two coupling fittings 175 and the lysate containing nucleic acid freed from cellular structures is transferred from vessel 170 to separation tube 176 as shown by the arrow in FIG. 4B. A magnet 180 is slidably coupled to tube 176 by a slider 181. In some embodiments, slider 181 is a conventional “zip tie” coupler. Use of a zip tie is by example only and not meant to be limiting. Any suitable sliding device may be used to slidable couple magnet 180 to separation tube 176 whereby magnet 180 may be freely moved between two ends of separation tube 176 while remaining proximate to a wall of separation tube 176, as shown in the several drawing figures. Magnet 180 movable along separation tube 176 via the action of slider 181 facilitates one or more serial washings of nucleic acid bound to paramagnetic beads and pelleted within separation tube 175, as discussed below.

FIGS. 5A-B are illustrations of a binding step. Binding step 325 comprises binding the nucleic acid for washing of protein, lipids, and other cellular contaminants present in the biological sample. In some embodiments, tube 176 is uncoupled from vessel 170 and coupled to a second tube (not shown) containing a volume of paramagnetic beads suspended in a binding buffer. The volume of suspended paramagnetic beads is transferred into separation tube 176 from the second tube coupled to separation tube 176 in a manner similar to transferring the diluent into vessel 170 in diluting step 315. Following transfer of the suspended paramagnetic beads into tube 176, the second tube is uncoupled from tube 176 and a small volume of air is drawn into tube 176 to create an air-fluid interface 177, as shown in FIG. 5B. The air transfer is effected by transferring air contained in an additional syringe coupled to tube 175, or drawn in from the environment, in some embodiments. Tube 176 is then coupled to a cap, a syringe, or other closing means at coupling fitting 175. Tube 176 is then allowed to sit undisturbed for a period of time (“incubation”) to allow binding of the nucleic acid to the paramagnetic beads, such as in a manner similar to method 100 discussed at length herein above. Following incubation, magnet 180 is slid across interface 177 to a position opposite the incubated fluid lysate-paramagnetic bead mixture, as shown in FIG. 5B. The paramagnetic particles bound to the nucleic acid are separated from solution after an additional period of incubation, as discussed herein above under the description of method 100.

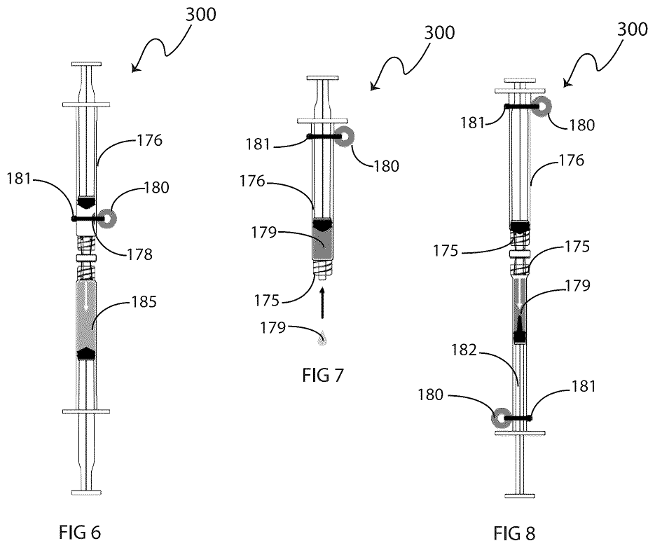

First removing step 330 comprises removing a first supernatant. FIG. 6 shows first removing step 330. A waste syringe 185 is coupled to tube 176 and a supernatant is drawn off of a bound nucleic acid 178. Attraction of the bound nucleic acid 178 on the paramagnetic beads attracted and fixed to the wall of tube 176 by magnet 189 retains nucleic acid 178 within tube 176, allowing removal of the supernatant, as shown.

FIG. 7 is an illustration of a washing step. Washing step 335 comprises washing the bound nucleic acid. FIG. 7 shows a wash buffer 179 being drawn through fitting 175 into tube 176. Wash buffer 179, in some embodiments, is contained within a syringe (not shown) coupled to fitting 175. After drawing wash buffer 179 into tube 176, magnet 180 is slid along tube 176 to a non-fluid containing region of tube 176, as shown in FIG. 7, releasing bound nucleic acid 178 from magnet 180 and allowing re-suspension of bound nucleic acid 178 in wash buffer 179.

FIG. 8 is an illustration of a second transferring step. Second transferring step 340 comprises transferring the bound nucleic acid. FIG. 8 shows a smaller tube 182 coupled to tube 176 at complementary fittings 175. Wash buffer 179 containing re-suspended bound nucleic acid 178 is transferred from tube 176 into smaller tube 182.

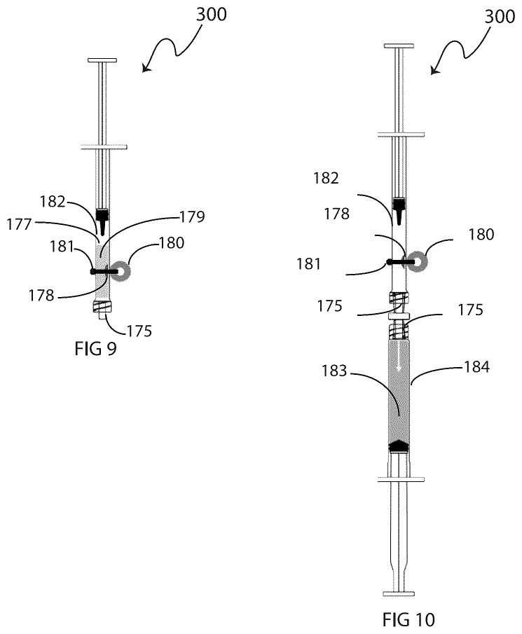

FIG. 9 is an illustration of a pelleting step. Pelleting step 345 comprises pelleting the bound nucleic acid. FIG. 9 shows pelleting step 345. Magnet 180 is slid along smaller tube 182 to a region opposite fluid contained within tube 182, as shown. The paramagnetic beads bearing bound nucleic acid 178 are pelleted against a region of a wall of tube 182 opposite magnet 180, following a period of incubation as discussed herein. After the incubation period, a volume of air is transferred into tube 176 to form interface 177, as shown.

FIG. 10 is an illustration of a second removing step. Second removing step 350 comprises removing a second supernatant. FIG. 10 shows separation tube 176 and a waste tube 184 coupled together at complementary fittings 175. Bound nucleic acid 178 remains fixed within separation tube 175 as a second supernatant 183 is drawn into waste tube 184, as shown.

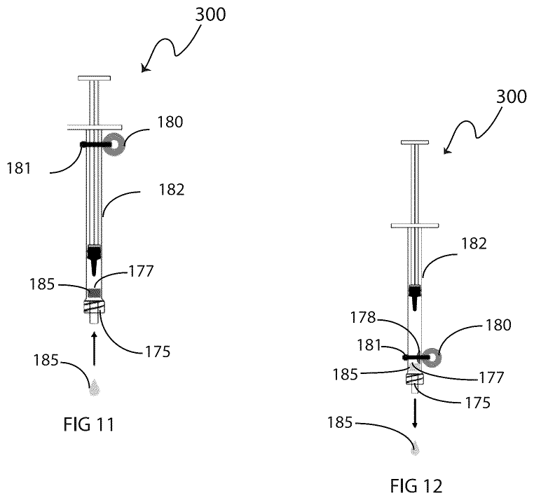

FIG. 11 is an illustration of a resuspending step. Resuspending step 355 comprises resuspending the bound nucleic acid. FIG. 11 shows resuspending step 355. A volume of elution buffer 185 is transferred into tube 176 from an additional tube (not shown) coupled to fitting 175. Magnet 180 is then slid into a position along smaller tube 182 away from elution buffer 185, as shown, to release and allow suspension of the pelleted bound nucleic acid 178 within elution buffer 185.

FIG. 12 is an illustration of an eluting step. Eluting step 360 comprises eluting a purified nucleic acid. FIG. 12 shows eluting step 360. In some embodiments, a small volume of elution buffer 185 is transferred into tube 182 through fitting 175. A volume of air is also transferred into tube 182 to create an interface 177 as shown in FIGS. 11-12. A washed and purified nucleic acid is then released and suspended into the small volume of elution buffer 186. Magnet 180 is then slid along smaller tube 182 to a region proximate to elution buffer 185 wherein the washed and purified nucleic acid. The paramagnetic beads, unbound from the washed and purified nucleic acid, and then fixed to the side of tube 182 proximate to magnet 180. The small volume of elution buffer 186 containing the washed and purified acid may then be transferred from tube 182 to a microcentrifuge tube via pipette tip for incorporation into a workflow that prepares DNA for sequencing (not shown) with the unbound paramagnetic beads remaining in tube 182 fixed by magnet 180 as shown in FIG. 12.

Some embodiments of the methods disclosed herein are executed using a kit containing the materials, reagents, and substances used to extract nucleic acid from a sample. In some embodiments, the kit includes the materials listed in Table 4A for processing with a PBS-based lysis buffer and Table 4B for a PBS-free NaCl-based lysis buffer:

TABLE 4A

| | |
|---|---|
| Material | Quantity |

| Lysis Buffer Vessel | | |
|---|---|---|
| 2 mL microcentrifuge tube containing: | | |
| 1,700 μL PBS | 4 | |
| 200 μL NaCl 5M (500 mM final concentration) | | |
| 100 μL Triton-X 10% (0.5% final concentration) | | |
| Wash Buffer Container | | |
| 15 mL conical tube containing: | | |
| 4 mL PEG 400 (5% final concentration) | 1 | |
| 8 mL NaCl 5M (500 mM final concentration) | | |
| 68 μL Nuclease-free Water | | |
| Lancet | 4 | |
| Hydrophilic Carrier | 4 | |
| 2 mL microcentrifuge tube containing lysis beads | 4 | |
| Nuclease-free water | 5 | mL |
| Paramagnetic Beads | 1 | mL |
| TE Buffer | 100 | μL |

TABLE 4B

| | |
|---|---|
| Material | Quantity |

| Lysis Buffer Vessel | | |
|---|---|---|
| 2 mL Eppendorf tube containing: | | |
| 1,800 μL NaCl 5M (4.5M final concentration) | 1 | |
| 200 μL Triton-X 10% (1% final concentration) | | |
| Wash Buffer Container | | |
| 2 mL microcentrifuge tube containing: | | |
| 400 uL PEG 3000 (10% final concentration) | 1 | |
| 200 uL NaCl 5M (500 mM final concentration) | | |
| 1400 μL Nuclease-free Water | | |
| Lancet | 4 | |
| Hydrophilic Carrier | 4 | |
| 2 mL microcentrifuge tube containing lysis beads | 4 | |
| Nuclease-free water | 1 | mL |
| Paramagnetic Beads | 800 | μL |
| TE Buffer | 100 | μL |

The values listed in the “Quantity” column of Tables 4A-B 1 are by way of example on only and not meant to be limiting. In some embodiments, to create a 2 mL solution of an alternate lysis buffer, an alternative nonionic surfactant is used in place of the Triton-X, the salt concentration can be adjusted to up to a 5 M final concentration to a final volume of 2 mL.

### Example Protocol Using Hydrophilic Polymer Foam as Carrier:

1. Use the lancet and allow blood to pool to a drop about 0.5 cm in diameter on the fingertip for 30 seconds. Massage the finger to force more blood out if needed.
2. With tweezers, soak up the pooled blood with an 8 mm×5 mm piece of absorbent hydrophilic polymer foam or 10 mm piece of filter paper.
3. Place the blood-soaked piece of foam or filter paper in the 2 mL Eppendorf tube that already contains 200 uL of extraction buffer and lysing beads.
4. Repeat collection for four (4) additional tubes if biological replicates are required.
5. Shake the tubes vigorously for 2 minutes to homogenize the blood.
6. Using a P200 pipette, transfer 100 uL of homogenized blood solution into a new 2 mL microcentrifuge tube. Avoid drawing debris into the pipet tip.
7. Using a P1000 pipette, add 900 uL of nuclease-free water.
8. Using a P1000 pipette, transfer 800 uL of diluted lysate into a new 2 mL tube.
9. Using a P1000 pipette, Add 640 uL of Ampure XP® paramagnetic beads.
10. Gently rock tube back and forth 20 times to mix contents.
11. Incubate at room temperature for 10 minutes.
12. Place on a magnet stand for 5 minutes to pellet the magnetic beads.
13. Carefully remove and discard the supernatant, ensuring that the beads are not disturbed.
14. Using a P1000 pipette, add 1 mL of wash buffer to the tube.
15. Resuspend the beads in the wash buffer by finger-flicking the tube repeatedly until the mixture homogenizes to a consistent brown color.
16. Let tube incubate at room temperature for 10 minutes.
17. Place on a magnet stand for 3 minutes to pellet the magnetic beads.
18. Using a new P1000 pipette carefully remove and discard the cleared wash buffer supernatant, ensuring that the bead pellet is not disturbed.
19. Remove any remnants of wash buffer with a P20 pipette.
20. Remove the tube from the magnet. Using a P20 pipette, add 22 uL of TE buffer and resuspend the beads completely by flicking the tube until the beads are fully emulsified.
21. Incubate at room temperature for 3 minutes, flicking the tube aggressively for 15 seconds every 45 seconds.
22. Place the tube back on the magnet and allow beads to pellet for 3 minutes.
23. Using a P20 pipette, transfer all 22 uL of supernatant to a new 1.5 mL tube. This is the eluted DNA.
24. If replicates were prepared, combine multiple elutions by pooling DNA and separate into 2 aliquots. One will be used for sequencing, the other will be brought back to the research team on Earth.

### EXAMPLE METHOD EMBODIMENTS AND RESULTS

Additional method embodiments example parameters of various wash and lysis buffer compositions, lysis times, paramagnetic bead ratios, and the like are shown in Tables 5-36 below. Also included are example gel electrophoresis results of the DNA generated by this method.

TABLE 5
Tris-based lysis buffer with Tris HCL and no Triton X-100
Lysis buffer with Tris HCl and no Triton-X
2 min. bead beating

| Lysis | | Vol | Wash | | Vol |
|---|---|---|---|---|---|
| buffer | Final C | (uL) | buffer | Final C | (uL) |
| NaCl 5M | 500 mM | 200 | NaCl | 0.5M | 1000 |
| Tris HCl | 10 mM | 1800 | PEG400 | 5% | 500 |
| | | | H2O | | 8500 |

| Blood | Lysis | Dilution | Beads | Wash | Elute |
|---|---|---|---|---|---|
| (uL) | (uL) | (uL) | (uL) | (uL) | (uL) |
| 40 | 200 | 800 | 1000 | 1000 | 10 |

| Sample | C (ng/uL) | V (uL) | DNA (ng) |
|---|---|---|---|
| A | 2.88 | 10 | 28.8 |
| B | 3.1 | 10 | 31.0 |
| C | 2.7 | 10 | 27.0 |

TABLE 6
Tris-based lysis buffer with Tris HCL and low concentration Triton X-100
Lysis buffer with Tris HCl and low Triton-X
2 min. bead beating

| Lysis | | Vol | Wash | | Vol |
|---|---|---|---|---|---|
| buffer | Final C | (uL) | buffer | Final C | (uL) |
| NaCl 5M | 500 mM | 200 | NaCl | 0.5M | 1000 |
| Triton X-100 | 0.10% | 20 | PEG400 | 5% | 500 |
| Tris HCl | 10 mM | 1780 | H2O | | 8500 |

| Blood | Lysis | Dilution | Beads | Wash | Elute |
|---|---|---|---|---|---|
| (uL) | (uL) | (uL) | (uL) | (uL) | (uL) |
| 40 | 200 | 800 | 1000 | 1000 | 10 |

| Sample | C (ng/uL) | V (uL) | DNA (ng) |
|---|---|---|---|
| A | 0.92 | 10 | 9.2 |
| B | 0.99 | 10 | 9.9 |
| C | 1.2 | 10 | 12.0 |

TABLE 7
PBS-based lysis buffer with PBS and no Triton X-100
Lysis buffer with PBS and no Triton-X
2 min. bead beating

| Lysis | | Vol | Wash | | Vol |
|---|---|---|---|---|---|
| buffer | Final C | (uL) | buffer | Final C | (uL) |
| NaCl 5M | 500 mM | 200 | NaCl | 0.5M | 1000 |
| PBS | 1X | 1800 | PEG400 | 5% | 500 |
| | | | H2O | | 8500 |

| Blood | Lysis | Dilution | Beads | Wash | Elute |
|---|---|---|---|---|---|
| (uL) | (uL) | (uL) | (uL) | (uL) | (uL) |
| 40 | 200 | 800 | 1000 | 1000 | 10 |

| Sample | C (ng/uL) | V (uL) | DNA (ng) |
|---|---|---|---|
| A | 1.62 | 10 | 16.2 |
| B | 2.46 | 10 | 24.6 |
| C | 1.88 | 10 | 18.8 |

TABLE 8
PBS-based lysis buffer with PBS and low concentration Triton X-100
Lysis buffer with PBS and low Triton-X
2 min. bead beating

| Lysis | | Vol | Wash | | Vol |
|---|---|---|---|---|---|
| buffer | Final C | (uL) | buffer | Final C | (uL) |
| NaCl 5M | 500 mM | 200 | NaCl | 0.5M | 1000 |
| Triton X-100 | 0.10% | 20 | PEG400 | 5% | 500 |
| PBS | 1X | 1780 | H2O | | 8500 |

| Blood | Lysis | Dilution | Beads | Wash | Elute |
|---|---|---|---|---|---|
| (uL) | (uL) | (uL) | (uL) | (uL) | (uL) |
| 40 | 200 | 800 | 1000 | 1000 | 10 |

| Sample | C (ng/uL) | V (uL) | DNA (ng) |
|---|---|---|---|
| A | 3.92 | 10 | 39.2 |
| B | 2.96 | 10 | 29.6 |
| C | 3.75 | 10 | 37.5 |

TABLE 9
PBS-based lysis buffer with water only
Lysis buffer with water only
2 min. bead beating

| Lysis | | Vol | Wash | | Vol |
|---|---|---|---|---|---|
| buffer | Final C | (uL) | buffer | Final C | (uL) |
| H2O | | 250 | NaCl | 0.5M | 1000 |
| | | | PEG400 | 5% | 500 |
| | | | H2O | | 8500 |

| Blood | Lysis | Dilution | Beads | Wash | Elute |
|---|---|---|---|---|---|
| (uL) | (uL) | (uL) | (uL) | (uL) | (uL) |
| 50 | 250 | 800 | 1000 | 1000 | 10 |

| Sample | C (ng/uL) | V (uL) | DNA (ng) |
|---|---|---|---|
| A | 1.58 | 11 | 17.4 |
| B | 1.4 | 11 | 15.4 |
| C | 1.6 | 11 | 17.6 |

TABLE 10
PBS-based lysis buffer with alternate salt formulation
Lysis buffer with alternate salt formulation
2 min. bead beating

| Lysis | | Vol | Wash | | Vol |
|---|---|---|---|---|---|
| buffer | Final C | (uL) | buffer | Final C | (uL) |
| Tris HCl 1M | 10 mM | 20 | NaCl | 0.5M | 1000 |
| KCl 2M | 10 mM | 10 | PEG400 | 5% | 500 |
| EDTA 0.5M | 2 mM | 8 | H2O | | 8500 |
| MgCl2 1M | 4 mM | 8 | | | |
| H2O | | 1954 | | | |

| Blood | Lysis | Dilution | Beads | Wash | Elute |
|---|---|---|---|---|---|
| (uL) | (uL) | (uL) | (uL) | (uL) | (uL) |
| 50 | 250 | 800 | 1000 | 1000 | 10 |

| Sample | C (ng/uL) | V (uL) | DNA (ng) |
|---|---|---|---|
| A | 0.78 | 11 | 8.6 |
| B | 0.6 | 11 | 6.6 |
| C | 0.81 | 11 | 8.9 |

TABLE 11
PBS-based lysis buffer with alternate salt formulation and Triton X-100
Lysis buffer with alternate salt formulation and Triton X-100
2 min. bead beating

| Lysis | | Vol | Wash | | Vol |
|---|---|---|---|---|---|
| buffer | Final C | (uL) | buffer | Final C | (uL) |
| Tris HCl 1M | 10 mM | 20 | NaCl | 0.5M | 1000 |
| KCl 2M | 10 mM | 10 | PEG400 | 5% | 500 |
| EDTA 0.5M | 2 mM | 8 | H2O | | 8500 |
| MgCl2 1M | 4 mM | 8 | | | |
| Triton X-100 10% | 0.50% | 100 | | | |
| H2O | | 1954 | | | |

| Blood | Lysis | Dilution | Beads | Wash | Elute |
|---|---|---|---|---|---|
| (uL) | (uL) | (uL) | (uL) | (uL) | (uL) |
| 50 | 250 | 800 | 1000 | 1000 | 10 |

| Sample | C (ng/uL) | V (uL) | DNA (ng) |
|---|---|---|---|
| A | 0.18 | 11 | 2.0 |
| B | 0 | 11 | 0.0 |
| C | 0.16 | 11 | 1.8 |

TABLE 12
PBS-based lysis buffer with only Triton X-100
Lysis buffer with only Triton X-100
2 min. bead beating

| Lysis | | Vol | Wash | | Vol |
|---|---|---|---|---|---|
| buffer | Final C | (uL) | buffer | Final C | (uL) |
| Triton X-100 10% | 1% | 200 | NaCl | 0.5M | 1000 |
| H2O | | 1800 | PEG400 | 5% | 500 |
| | | | H2O | | 8500 |

| Blood | Lysis | Dilution | Beads | Wash | Elute |
|---|---|---|---|---|---|
| (uL) | (uL) | (uL) | (uL) | (uL) | (uL) |
| 50 | 250 | 800 | 1000 | 1000 | 10 |

| Sample | C (ng/uL) | V (uL) | DNA (ng) |
|---|---|---|---|
| A | 0.56 | 11 | 6.2 |
| B | 0.3 | 11 | 3.3 |
| C | 0.62 | 11 | 6.8 |

TABLE 13
Regular salt wash buffer
PBS-based lysis, 2 min. bead beating

| Lysis | | Vol | Wash | | Vol |
|---|---|---|---|---|---|
| buffer | Final C | (uL) | buffer | Final C | (uL) |
| NaCl 5M | 500 mM | 200 | NaCl | 1.25M | 2500 |
| Triton-X 10% | 0.50% | 100 | PEG400 | 5% | 500 |
| PBS 1X | | | H2O | | 7000 |

| Blood | Lysis | Dilution | Beads | Wash | Elute |
|---|---|---|---|---|---|
| (uL) | (uL) | (uL) | (uL) | (uL) | (uL) |
| 20 | 200 | 800 | 1000 | 1000 | 10 |

| Sample | C (ng/uL) | V (uL) | DNA (ng) |
|---|---|---|---|
| A | 9.2 | 10 | 92 |
| B | 8.85 | 10 | 88.5 |
| C | 9.33 | 10 | 93.3 |

TABLE 14
Low salt wash buffer
PBS-based lysis, 2 min. bead beating

| Lysis | | Vol | Wash | | Vol |
|---|---|---|---|---|---|
| buffer | Final C | (uL) | buffer | Final C | (uL) |
| NaCl 5M | 500 mM | 200 | NaCl | 0.5M | 1000 |
| Triton-X 10% | 0.50% | 100 | PEG400 | 5% | 500 |
| PBS 1X | | | H2O | | 8500 |

| Blood | Lysis | Dilution | Beads | Wash | Elute |
|---|---|---|---|---|---|
| (uL) | (uL) | (uL) | (uL) | (uL) | (uL) |
| 20 | 200 | 800 | 1000 | 1000 | 10 |

| Sample | C (ng/uL) | V (uL) | DNA (ng) |
|---|---|---|---|
| A | 8.56 | 10 | 85.6 |
| B | 9.1 | 10 | 91 |
| C | 8.3 | 10 | 83 |

TABLE 15
Very low salt wash buffer
PBS-based lysis, 2 min. bead beating

| Lysis | | Vol | Wash | | Vol |
|---|---|---|---|---|---|
| buffer | Final C | (uL) | buffer | Final C | (uL) |
| NaCl 5M | 500 mM | 200 | NaCl | 0.1M | 200 |
| Triton-X 10% | 0.50% | 100 | PEG400 | 5% | 500 |
| PBS 1X | | | H2O | | 9300 |

| Blood | Lysis | Dilution | Beads | Wash | Elute |
|---|---|---|---|---|---|
| (uL) | (uL) | (uL) | (uL) | (uL) | (uL) |
| 20 | 200 | 800 | 1000 | 1000 | 10 |

| Sample | C (ng/uL) | V (uL) | DNA (ng) |
|---|---|---|---|
| A | 2.14 | 18 | 38.5 |
| B | 1.91 | 18 | 34.4 |
| C | 1.85 | 18 | 33.3 |

TABLE 16
Mechanical lysis with pestle and water only
2 min. pestle grinding

| Lysis | Vol | Wash | | Vol |
|---|---|---|---|---|
| buffer | (uL) | buffer | Final C | (uL) |
| H2O | 2000 | NaCl | 1.25M | 2500 |
| | | PEG400 | 5% | 500 |
| | | H2O | | 7000 |

| Blood | Lysis | Dilution | Beads | Wash | Elute |
|---|---|---|---|---|---|
| (uL) | (uL) | (uL) | (uL) | (uL) | (uL) |
| 50 | 250 | 800 | 1000 | 1000 | 10 |

| Sample | C (ng/uL) | V (uL) | DNA (ng) |
|---|---|---|---|
| A | 0.23 | 11 | 2.5 |
| B | 0.96 | 11 | 10.6 |
| C | 0.61 | 11 | 6.7 |

TABLE 17
Diluted lysate with 800 uL 1X PBS after lysis
PBS-based lysis, 2 min. bead beating

| Lysis | Final | Vol | Wash | Final | Vol |
|---|---|---|---|---|---|
| buffer | C | (uL) | buffer | C | (uL) |
| NaCl 5M | 500 mM | 200 | NaCl | 1.25M | 2500 |
| Triton-X 10% | 0.50% | 100 | PEG400 | 5% | 500 |
| PBS 1X | | | H2O | | 7000 |

| Blood | Lysis | Dilution | Beads | Wash | Elute |
|---|---|---|---|---|---|
| (uL) | (uL) | (uL) | (uL) | (uL) | (uL) |
| 20 | 200 | 800 | 1000 | 1000 | 10 |

| | C | V | DNA |
|---|---|---|---|
| Sample | (ng/uL) | (uL) | (ng) |
| A | 9.9 | 10 | 99 |
| B | 10.1 | 10 | 101 |
| C | 9.2 | 10 | 92 |

TABLE 18
Diluted lysate with 800 ul water after lysis
PBS-based lysis, 2 min. bead beating

| Lysis | Final | Vol | Wash | Final | Vol |
|---|---|---|---|---|---|
| buffer | C | (uL) | buffer | C | (uL) |
| NaCl 5M | 500 mM | 200 | NaCl | 0.5M | 1000 |
| Triton-X 10% | 0.50% | 100 | PEG400 | 5% | 500 |
| PBS 1X | | | H2O | | 8500 |

| Blood | Lysis | Dilution | Beads | Wash | Elute |
|---|---|---|---|---|---|
| (uL) | (uL) | (uL) | (uL) | (uL) | (uL) |
| 20 | 200 | 800 | 1000 | 1000 | 10 |

| | C | V | DNA |
|---|---|---|---|
| Sample | (ng/uL) | (uL) | (ng) |
| A | 10.2 | 10 | 102 |
| B | 9.65 | 10 | 96.5 |
| C | 10.3 | 10 | 103 |

TABLE 19
Using filter paper as blood carrier
PBS-based lysis, 2 min. bead beating

| Lysis | Final | Vol | Wash | Final | Vol |
|---|---|---|---|---|---|
| buffer | C | (uL) | buffer | C | (uL) |
| NaCl 5M | 500 mM | 200 | NaCl | 1.25M | 2500 |
| Triton-X 10% | 0.50% | 100 | PEG400 | 5% | 500 |
| PBS 1X | | | H2O | | 7000 |

| Blood | Lysis | Dilution | Beads | Wash | Elute |
|---|---|---|---|---|---|
| (uL) | (uL) | (uL) | (uL) | (uL) | (uL) |
| 20 | 200 | 800 | 1000 | 1000 | 10 |

| | C | V | DNA |
|---|---|---|---|
| Sample | (ng/uL) | (uL) | (ng) |
| A | 9.85 | 10 | 98.5 |
| B | 9.32 | 10 | 93.2 |
| C | 9.5 | 10 | 95 |

TABLE 20
Using hydrophilic polymer foam as blood carrier
PBS-based lysis, 2 min. bead beating

| Lysis | Final | Vol | Wash | Final | Vol |
|---|---|---|---|---|---|
| buffer | C | (uL) | buffer | C | (uL) |
| NaCl 5M | 500 mM | 200 | NaCl | 0.5M | 1000 |
| Triton-X 10% | 0.50% | 100 | PEG400 | 5% | 500 |
| PBS 1X | | | H2O | | 8500 |

| Blood | Lysis | Dilution | Beads | Wash | Elute |
|---|---|---|---|---|---|
| (uL) | (uL) | (uL) | (uL) | (uL) | (uL) |
| 20 | 200 | 800 | 1000 | 1000 | 10 |

| | C | V | DNA |
|---|---|---|---|
| Sample | (ng/uL) | (uL) | (ng) |
| A | 10.2 | 10 | 102 |
| B | 12.1 | 10 | 121 |
| C | 11.5 | 10 | 115 |

TABLE 21
Adding blood directly to tube without a carrier
PBS-based lysis, 2 min. bead beating

| Lysis | Final | Vol | Wash | Final | Vol |
|---|---|---|---|---|---|
| buffer | C | (uL) | buffer | C | (uL) |
| NaCl 5M | 500 mM | 200 | NaCl | 0.5M | 1000 |
| Triton-X 10% | 0.50% | 100 | PEG400 | 5% | 500 |
| PBS 1X | | | H2O | | 8500 |

| Blood | Lysis | Dilution | Beads | Wash | Elute |
|---|---|---|---|---|---|
| (uL) | (uL) | (uL) | (uL) | (uL) | (uL) |
| 20 | 200 | 800 | 1000 | 1000 | 10 |

| | C | V | DNA |
|---|---|---|---|
| Sample | (ng/uL) | (uL) | (ng) |
| A | 1.4 | 10 | 14 |
| B | 0.93 | 10 | 9.3 |
| C | 0.71 | 10 | 7.1 |

TABLE 22
Ecosurf EH-9 substitution for Triton X-100 trials
Determine effect of substituting Ecosurf EH-9 for
Triton X-100 in lysis buffer
2 min. bead beating (BB), 0.8x SPRI cleanup with 10
min. incubations, Resuspension during wash

| | Blood:LB | Blood | LB | | BB duration |
|---|---|---|---|---|---|
| Sample | ratio | V (uL) | V (uL) | Beads | (min) |
| A | 1:4 | 50 | 200 | Yes | 2 |

| Lysis | Final | Vol | Wash | Final | Vol |
|---|---|---|---|---|---|
| buffer (LB) | C | (uL) | buffer (WB) | C | (uL) |
| NaCl 5M | 4.5M | 1800 | NaCl 5M | 0.5M | 200 |
| Ecosurf EH-9 10% | 1% | 200 | PEG 3000 50% | 10% | 400 |
| | | | H2O | | 1400 |

| Lysate | H2O | Diluted | SPRI | Wash | Elute |
|---|---|---|---|---|---|
| (uL) | (uL) | lysate (uL) | Beads (uL) | (uL) | (uL) |
| 100 | 900 | 800 | 640 | 1000 | 22 |

| | C | V | DNA | | |
|---|---|---|---|---|---|
| Sample | (ng/uL) | (uL) | (ng) | A260/280 | Appearance |
| A1 | 9.9 | 20 | 198.8 | 1.49 | Clear |
| A2 | 10.3 | 20 | 206 | 1.48 | Clear |
| A3 | 10.1 | 20 | 202 | 1.55 | Clear |

TABLE 23
Mechanical lysis bead beating validation
Determine effect of removing bead beating (BB) during lysis
2 min shaking during lysis with and without beads, 0.8x SPRI
cleanup with 10 min. incubations, Resuspension during wash

| | Blood:LB | Blood | LB | | Shaking |
|---|---|---|---|---|---|
| Sample | ratio | V (uL) | V (uL) | Beads | (min) |
| A (No beads) | 1:4 | 50 | 200 | No | 2 |
| B (Beads) | 1:4 | 50 | 200 | Yes | 2 |

| Lysis | Final | Vol | Wash | Final | Vol |
|---|---|---|---|---|---|
| buffer (LB) | C | (uL) | buffer (WB) | C | (uL) |
| NaCl 5M | 4.5M | 1800 | NaCl 5M | 0.5M | 200 |
| Triton X-100 10% | 1% | 200 | PEG 3000 50% | 10% | 400 |
| | | | H2O | | 1400 |

| Lysate | H2O | Diluted | SPRI | Wash | Elute |
|---|---|---|---|---|---|
| (uL) | (uL) | lysate (uL) | Beads (uL) | (uL) | (uL) |
| 100 | 900 | 800 | 640 | 1000 | 22 |

| | C | V | DNA | | |
|---|---|---|---|---|---|
| Sample | (ng/uL) | (uL) | (ng) | A260/280 | Appearance |
| A1 | 8.2 | 20 | 164.4 | 1.14 | Clear |
| A2 | 7.8 | 20 | 156 | 1.13 | Clear |
| A3 | 7.5 | 20 | 150 | 1.1 | Clear |
| B1 | 10.8 | 20 | 215.2 | 1.49 | Clear |
| B2 | 10.6 | 20 | 211.6 | 1.52 | Clear |
| B3 | 10.2 | 20 | 204.8 | 1.53 | Clear |

TABLE 24
Mechanical lysis bead beating duration test
Determine effect of reducing bead beating (BB) duration
1 min bead beating (BB), 0.8x SPRI cleanup with 10 min.
incubations, Resuspension during wash

| | Blood:LB | Blood | LB | | BB duration |
|---|---|---|---|---|---|
| Sample | ratio | V (uL) | V (uL) | Beads | (min) |
| A (1 min BB) | 1:4 | 50 | 200 | Yes | 1 |

| Lysis | Final | Vol | Wash | Final | Vol |
|---|---|---|---|---|---|
| buffer (LB) | C | (uL) | buffer (WB) | C | (uL) |
| NaCl 5M | 4.5M | 1800 | NaCl 5M | 0.5M | 200 |
| Triton X-100 10% | 1% | 200 | PEG 3000 50% | 10% | 400 |
| | | | H2O | | 1400 |

| Lysate | H2O | Diluted | SPRI | Wash | Elute |
|---|---|---|---|---|---|
| (uL) | (uL) | lysate (uL) | Beads (uL) | (uL) | (uL) |
| 100 | 900 | 800 | 640 | 1000 | 22 |

| | C | V | DNA | | |
|---|---|---|---|---|---|
| Sample | (ng/uL) | (uL) | (ng) | A260/280 | Appearance |
| A1 | 10.1 | 20 | 202 | 1.48 | Clear |
| A2 | 11 | 20 | 220 | 1.5 | Clear |
| A3 | 10.4 | 20 | 208 | 1.52 | Clear |

TABLE 25
Post-lysis dilution validation
Determine effect of removing dilution step
2 min. bead beating (BB), 0.8x SPRI cleanup with
10 min. incubations, Resuspension during wash

| | Blood:LB | Blood | LB | | BB duration |
|---|---|---|---|---|---|
| Sample | ratio | V (uL) | V (uL) | Beads | (min) |
| A | 1:4 | 50 | 200 | Yes | 2 |

| Lysis | Final | Vol | Wash | Final | Vol |
|---|---|---|---|---|---|
| buffer (LB) | C | (uL) | buffer (WB) | C | (uL) |
| NaCl 5M | 4.5M | 1800 | NaCl 5M | 0.5M | 200 |
| Triton X-100 10% | 1% | 200 | PEG 3000 50% | 10% | 400 |
| | | | H2O | | 1400 |

| Lysate | H2O | Diluted | SPRI | Wash | Elute |
|---|---|---|---|---|---|
| (uL) | (uL) | lysate (uL) | Beads (uL) | (uL) | (uL) |
| 200 | 0 | 200 | 160 | 1000 | 22 |

| | C | V | DNA | | |
|---|---|---|---|---|---|
| Sample | (ng/uL) | (uL) | (ng) | A260/280 | Appearance |
| A1 | 18.9 | 20 | 378 | 0.73 | Foamy |
| A2 | 22.8 | 20 | 456 | 0.69 | Foamy |
| A3 | 20.9 | 20 | 418 | 0.67 | Foamy |

TABLE 26
SPRI bead ratio testing
Test bead ratios in SPRI bead purification step
2 min. bead beating (BB), 0.8x SPRI cleanup with
10 min. incubations, with wash resuspension

| | Blood:LB | Blood | LB | | BB duration |
|---|---|---|---|---|---|
| Sample | ratio | V (uL) | V (uL) | Beads | (min) |
| A (0.8x) | 1:4 | 50 | 200 | Yes | 2 |
| B (1.2x) | 1:4 | 50 | 200 | Yes | 2 |

| Lysis | Final | Vol | Wash | Final | Vol |
|---|---|---|---|---|---|
| buffer (LB) | C | (uL) | buffer (WB) | C | (uL) |
| NaCl 5M | 4.5M | 1800 | NaCl 5M | 0.5M | 200 |
| Triton X-100 10% | 1% | 200 | PEG 3000 50% | 10% | 400 |
| | | | H2O | | 1400 |

| Lysate | H2O | Diluted | SPRI | Wash | Elute |
|---|---|---|---|---|---|
| (uL) | (uL) | lysate (uL) | Beads (uL) | (uL) | (uL) |
| 100 | 900 | 800 | 640, 960 | 1000 | 22 |

| | C | V | DNA | | |
|---|---|---|---|---|---|
| Sample | (ng/uL) | (uL) | (ng) | A260/280 | Condition |
| A1 | 11.5 | 20 | 230 | 1.5 | 0.8x |
| A2 | 10.9 | 20 | 218 | 1.52 | 0.8x |
| A3 | 12.1 | 20 | 242 | 1.56 | 0.8x |
| B1 | 15.7 | 20 | 314 | 1.49 | 1.2x |
| B2 | 14.6 | 20 | 292 | 1.46 | 1.2x |
| B3 | 16.1 | 20 | 322 | 1.53 | 1.2x |

TABLE 27
Wash buffer salt content testing
Test effect of varying salt concentration in wash buffer
2 min. bead beating (BB), 0.8x SPRI cleanup with 10 min.
incubations, with or without wash resuspension

| | Blood:LB | Blood | LB | | BB duration |
|---|---|---|---|---|---|
| Sample | ratio | V (uL) | V (uL) | Beads | (min) |
| A (0.35M NaCl) | 1:4 | 50 | 200 | Yes | 2 |
| B (0.5M NaCl) | 1:4 | 50 | 200 | Yes | 2 |
| C (1M NaCl) | 1:4 | 50 | 200 | Yes | 2 |

| Lysis | Final | Vol | Wash | Final | Vol |
|---|---|---|---|---|---|
| buffer (LB) | C | (uL) | buffer (WB) | C | (uL) |
| NaCl 5M | 4.5M | 1800 | NaCl 5M | 0.35M, | 140, 200, 400 |
| | | | | 0.5M, 1M | |
| Triton X-100 10% | 1% | 200 | PEG 3000 50% | 10% | 400 |
| | | | H2O | | 1400 |

| Lysate | H2O | Diluted | SPRI | Wash | Elute |
|---|---|---|---|---|---|
| (uL) | (uL) | lysate (uL) | Beads (uL) | (uL) | (uL) |
| 100 | 900 | 800 | 640 | 1000 | 22 |

| | C | V | DNA | | | |
|---|---|---|---|---|---|---|
| Sample | (ng/uL) | (uL) | (ng) | A260/280 | Condition | |
| A1 | 8.7 | 20 | 173.2 | 1.22 | 0.35M | NaCl |
| A2 | 6.3 | 20 | 126.8 | 1.26 | 0.35M | NaCl |
| A3 | 8.2 | 20 | 164 | 1.19 | 0.35M | NaCl |
| B1 | 12.2 | 20 | 244 | 1.53 | 0.5M | NaCl |
| B2 | 12.9 | 20 | 258 | 1.5 | 0.5M | NaCl |
| B3 | 11.7 | 20 | 234 | 1.59 | 0.5M | NaCl |
| C1 | 16.3 | 20 | 326 | 1.51 | 1M | NaCl |
| C2 | 14.6 | 20 | 292 | 1.46 | 1M | NaCl |
| C3 | 15.4 | 20 | 308 | 1.5 | 1M | NaCl |

TABLE 28
Wash buffer PEG content testing
Test effect of varying polyethylene glycol (PEG) concentration
in wash buffer
2 min. bead beating (BB), 0.8x SPRI cleanup with 10 min.
incubations, with or without wash resuspension

| | Blood:LB | Blood | LB | | BB duration |
|---|---|---|---|---|---|
| Sample | ratio | V (uL) | V (uL) | Beads | (min) |
| A (5% PEG) | 1:4 | 50 | 200 | Yes | 2 |
| B (10% PEG) | 1:4 | 50 | 200 | Yes | 2 |
| B (15% PEG) | 1:4 | 50 | 200 | Yes | 2 |

| Lysis | Final | Vol | Wash | Final | Vol |
|---|---|---|---|---|---|
| buffer (LB) | C | (uL) | buffer (WB) | C | (uL) |
| NaCl 5M | 4.5M | 1800 | NaCl 5M | 0.5M | 200 |
| Triton X-100 10% | 1% | 200 | PEG 3000 50% | 5%, 10%, | 200, 400, 600 |
| | | | | 15% | |
| | | | H2O | | 1400 |

| Lysate | H2O | Diluted | SPRI | Wash | Elute |
|---|---|---|---|---|---|
| (uL) | (uL) | lysate (uL) | Beads (uL) | (uL) | (uL) |
| 100 | 900 | 800 | 640 | 1000 | 22 |

| | C | V | DNA | | |
|---|---|---|---|---|---|
| Sample | (ng/uL) | (uL) | (ng) | A260/280 | Condition |
| A1 | 1.7 | 20 | 34.4 | 1.33 | 5% PEG |
| A2 | 2.1 | 20 | 42.8 | 1.27 | 5% PEG |
| A3 | 2.0 | 20 | 39 | 1.29 | 5% PEG |
| B1 | 11.7 | 20 | 234 | 1.46 | 10% PEG |
| B2 | 13.1 | 20 | 262 | 1.49 | 10% PEG |
| B3 | 11.9 | 20 | 238 | 1.54 | 10% PEG |
| C1 | 6.6 | 20 | 132 | 1.2 | 15% PEG |
| C2 | 8.5 | 20 | 170 | 1.25 | 15% PEG |
| C3 | 7.9 | 20 | 157.2 | 1.26 | 15% PEG |

TABLE 29
SPRI bead incubation time tests
Determine effect of reducing SPRI bead incubation time
2 min. bead beating (BB), 0.8x SPRI cleanup with 5- or
10-min. incubations, Resuspension during wash

| | Blood:LB | Blood | LB | | BB duration |
|---|---|---|---|---|---|
| Sample | ratio | V (uL) | V (uL) | Beads | (min) |
| A (5 min inc) | 1:4 | 50 | 200 | Yes | 2 |
| B (10 min inc) | 1:4 | 50 | 200 | Yes | 2 |

| Lysis | Final | Vol | Wash | Final | Vol |
|---|---|---|---|---|---|
| buffer (LB) | C | (uL) | buffer (WB) | C | (uL) |
| NaCl 5M | 4.5M | 1800 | NaCl 5M | 0.5M | 200 |
| Triton X-100 10% | 1% | 200 | PEG 3000 50% | 10% | 400 |
| | | | H2O | | 1400 |

| Lysate | H2O | Diluted | SPRI | Wash | Elute |
|---|---|---|---|---|---|
| (uL) | (uL) | lysate (uL) | Beads (uL) | (uL) | (uL) |
| 100 | 900 | 800 | 640 | 1000 | 22 |

| | C | V | DNA | | |
|---|---|---|---|---|---|
| Sample | (ng/uL) | (uL) | (ng) | A260/280 | Appearance |
| A1 | 6.5 | 20 | 130.8 | 0.97 | 5 min incubation |
| A2 | 5.6 | 20 | 111.2 | 1.09 | 5 min incubation |
| A3 | 5.8 | 20 | 116 | 1.03 | 5 min incubation |
| B1 | 11 | 20 | 220 | 1.46 | 10 min incubation |
| B2 | 10.2 | 20 | 204 | 1.53 | 10 min incubation |
| B3 | 10.4 | 20 | 207.6 | 1.56 | 10 min incubation |

TABLE 30
SPRI bead resuspension validation
Determine need for SPRI bead resuspension during wash step
2 min. bead beating (BB), 0.8x SPRI cleanup with 10 min.
incubations, with or without wash resuspension

| | Blood:LB | Blood | LB | | BB duration |
|---|---|---|---|---|---|
| Sample | ratio | V (uL) | V (uL) | Beads | (min) |
| A (No resuspension) | 1:4 | 50 | 200 | Yes | 2 |
| B (Resuspension) | 1:4 | 50 | 200 | Yes | 2 |

| Lysis | Final | Vol | Wash | Final | Vol |
|---|---|---|---|---|---|
| buffer (LB) | C | (uL) | buffer (WB) | C | (uL) |
| NaCl 5M | 4.5M | 1800 | NaCl 5M | 0.5M | 200 |
| Triton X-100 10% | 1% | 200 | PEG 3000 50% | 10% | 400 |
| | | | H2O | | 1400 |

| Lysate | H2O | Diluted | SPRI | Wash | Elute |
|---|---|---|---|---|---|
| (uL) | (uL) | lysate (uL) | Beads (uL) | (uL) | (uL) |
| 100 | 900 | 800 | 640 | 1000 | 22 |

| | C | V | DNA | | |
|---|---|---|---|---|---|
| Sample | (ng/uL) | (uL) | (ng) | A260/280 | Appearance |
| A1 | 12.9 | 20 | 258 | 0.84 | Slightly pink |
| A2 | 12.3 | 20 | 246 | 0.86 | Slightly pink |
| A3 | 12.1 | 20 | 242 | 0.81 | Slightly pink |
| B1 | 10.5 | 20 | 210 | 1.52 | Clear |
| B2 | 11 | 20 | 220 | 1.56 | Clear |
| B3 | 10.8 | 20 | 216 | 1.49 | Clear |

TABLE 31
Syringe extraction trials
Testing effect of varying Triton X-100 (TX) concentration, bead ratio
(SPRI), salt content in wash buffer (WB) and elution volume
2 min. bead beating (BB), variable SPRI cleanup with 10 min.
incubations, with wash resuspension

| | Blood:LB | Blood | LB | | BB duration |
|---|---|---|---|---|---|
| Sample | ratio | V (uL) | V (uL) | Beads | (min) |
| A | 1:4 | 50 | 200 | Yes | 2 |

| Lysis | Final | Vol | Wash | Final | Vol |
|---|---|---|---|---|---|
| buffer (LB) | C | (uL) | buffer (WB) | C | (uL) |
| NaCl 5M | 4.5M | 1800 | NaCl 5M | 0.5M, 1M | 200, 400 |
| Triton X-100 10% | 0.5%, 1% | 100, 200 | PEG 3000 50% | 10% | 400 |
| | | | H2O | | 1400 |

| Lysate | H2O | Diluted | SPRI | Wash | Elute |
|---|---|---|---|---|---|
| (uL) | (uL) | lysate (uL) | Beads (uL) | (uL) | (uL) |
| 100 | 900 | 800 | 800, 960 | 500 | 100, 50 |

| | C | V | DNA | | |
|---|---|---|---|---|---|
| Sample | (ng/uL) | (uL) | (ng) | A260/280 | Condition |
| A1 | 2.9 | 100 | 292 | 1.48 | 1x SPRI, 0.5% TX |
| A2 | 6.4 | 50 | 318 | 1.52 | 1x SPRI, 0.5% TX |
| A3 | 6.7 | 50 | 335 | 1.56 | 1.2x SPRI, 1% TX |
| A4 | 7.6 | 50 | 378 | 1.48 | 1.2x SPRI, 1% TX, 1M NaCl |

Several embodiments of a simplified method of nucleic acid extraction have been presented. The methods allow for extraction of nucleic acids from a biological sample without toxic compositions, flammable solvents, the need for an external heat source, centrifugation, or electrical equipment. The methods can be performed effectively by personnel not skilled in laboratory techniques, in remote locations, and within limited space. The methods and kits described herein are proven by experimentation to provide sufficient yields of DNA from a sample of human whole blood suitable for sequencing and can be used to monitor astronauts for genetic mutations and epigenetic changes during prolonged time in outer space.

The embodiments and examples set forth herein were presented in order to best explain the present invention and its practical application, and to thereby enable those of ordinary skill in the art to make and use the invention. However, those of ordinary skill in the art will recognize that the foregoing description and examples have been presented for the purpose of illustration and example. The description as set forth is not intended to be exhaustive or to limit the invention to the precise form disclosed. Many modifications and variations are possible, in light of the teachings herein above.

What is claimed is:

1. A simplified method of nucleic acid extraction from a biological sample comprising steps:
collecting a biological sample;
preparing the sample;
lysing the sample to create a lysate containing a nucleic acid in the absence of an organic solvent;
fixing the nucleic acid;
washing the lysate from the fixed nucleic acid; and
eluting the nucleic acid, wherein the collecting step, the preparing step, and fixing step, the washing step, and the eluting step are performed without the use of electrically powered equipment and in the absence of an organic solvent.

2. A method of nucleic acid extraction from a biological sample comprising steps:
lysing a biological sample containing a nucleic acid to form a lysate;
diluting the lysate;
transferring the lysate;
binding the nucleic acid to a paramagnetic bead;
removing a first supernatant;
washing the bound nucleic acid;
transferring the bound nucleic acid;
pelleting the bound nucleic acid;
removing a second supernatant;
resuspending the bound nucleic acid; and
eluting the nucleic acid from the paramagnetic bead;
wherein the collecting step, the lysing step, the fixing step, the washing step, and the eluting step are performed without the use of electrically powered equipment and in the absence of an organic solvent.
